(19) United States
(12) Patent Application Publication
(10) Pub. No.: US 2015/0297475 A1
(43) Pub. Date: Oct. 22, 2015

(54) CARBON-DIOXIDE-GENERATING COSMETIC SHEET PRODUCT

(71) Applicant: Medion Research Laboratories Inc., Osaka (JP)

(72) Inventor: Masato HIKI, Osaka (JP)

(21) Appl. No.: 14/382,290

(22) PCT Filed: Feb. 26, 2013
(86) PCT No.: PCT/JP2013/054995

§ 371 Date: Aug. 29, 2014

(30) Foreign Application Priority Data
Feb. 29, 2012 (JP) 2012-044413

(51) Int. Cl. A61K 8/19; A61K 8/362; A61K 8/73; A61K 47/38; A61Q 19/02; A61K 9/70; A61K 33/00; A61K 47/12; A61K 8/02; A61Q 19/00

(52) U.S. Cl. CPC A61K 8/19; A61K 8/0208; A61K 8/362; A61K 8/731; A61Q 19/00; A61Q 19/02; A61K 9/70; A61K 33/00; A61K 47/12; A61K 47/38; A61K 2800/22; A61K 2800/87

(57) ABSTRACT

[Problem] The objective of the present invention is, in a cosmetic sheet product containing an acid-containing solution and a sheet substrate supporting a carbonate, to provide a formulation technology that uniformly and continuously causes the generation of minute bubbles of carbon dioxide.

[Solution] In the cosmetic sheet product containing an acid-containing solution and a sheet substrate supporting a carbonate, by means of admixing a hydrophobically modified alkyl cellulose to the acid-containing solution, it is possible to provide a cosmetic sheet product that can uniformly and continuously generate carbon dioxide in the form of minute bubbles and to enjoy the effect of carbon dioxide when the solution is caused to impregnate the sheet substrate.

### TECHNICAL FIELD

The present invention relates to a carbon-dioxide-generating cosmetic sheet product. More particularly, the present invention relates to a cosmetic sheet product including a solution (I) containing an acid, and a sheet substrate an carrying a carbonate, wherein minute bubbles of carbon dioxide can be uniformly and continuously generated when the sheet substrate (II) is impregnated with the solution (I).

### BACKGROUND ART

In recent years, consciousness about cosmetic has been increased regardless of sex, and various functional cosmetics have been developed in response to this trend. Among them, cosmetics focusing on efficacy of carbon dioxide receive great attention. Carbon dioxide has been heretofore known to have a blood circulation-promoting effect, and it has been reported in Patent Document 1 that by using a carbon-dioxide-containing water-containing viscous composition with carbon dioxide efficiently confined therein, cosmetic effects such as improvement of skin quality, skin whitening, amelioration of flabbiness, amelioration of darkening and slimming, and therapeutic effects for skin disease/injury, dental disease, musculoskeletal disease and neurological disease are obtained.

Patent Document 1 discloses various forms of kits for obtaining a carbon-dioxide-containing composition with carbon dioxide efficiently confined therein. Particularly, Patent Document 1 discloses a combination of an acid-containing water-containing viscous composition and a carbonate-containing sheet, wherein by bringing the composition and the sheet into contact with each other at the time of use, carbon dioxide can be generated. In Patent Document 1, however, an acid-containing water containing viscous composition in the form of a gel is used, but the combination is not used with the carbonate-containing sheet impregnated with a solution containing an acid.

A pack cosmetic capable of continuously generating carbon dioxide with a sheet substrate impregnated with a solution is expected to be able to meet consumers' demands that have been diversified in recent years. However, when a sheet substrate carrying a carbonate is impregnated with a solution containing an acid, there is the problem that carbon dioxide in the form of large bubbles is generated, and volatilization occurs immediately after carbon dioxide is generated from the sheet substrate.

Thus, in conventional techniques, a formulation technique has not been developed which allows minute bubbles of carbon dioxide to be uniformly and continuously generated in a cosmetic sheet product including a solution containing an acid and a sheet substrate carrying a carbonate, and in the present state, a cosmetic sheet capable of sufficiently exploiting efficacy of carbon dioxide cannot be provided.

### PRIOR ART DOCUMENT

### Patent Document

Patent Document 1: International Publication No. WO 99/24043

### SUMMARY OF THE INVENTION

### Problems to be Solved by the Invention

The object of the present invention is to provide a formulation technique which allows minute bubbles of carbon dioxide to be uniformly and continuously generated in a cosmetic sheet product including a solution containing an acid and a sheet substrate carrying a carbonate.

### Means for Solving the Problem

The present inventors have extensively conducted studies for solving the above-described problem, and resultantly found that by blending a hydrophobically modified alkyl cellulose in a solution containing an acid in a cosmetic sheet product including a solution containing an acid and a sheet substrate carrying a carbonate, carbon dioxide in the form of minute bubbles can be uniformly and continuously generated when the sheet substrate is impregnated with the solution, so that a cosmetic sheet product enabling enjoyment of efficacy of carbon dioxide can be provided. The present invention has been completed by further conducting studies based on the above-described findings.

That is, the present invention provides inventions having the aspect described below.

Item 1. A cosmetic sheet product including a solution (I) containing a hydrophobically modified alkyl cellulose, an acid and water; and a sheet substrate (II) carrying a carbonate and being used with the sheet substrate (II) impregnated with the solution (I).
Item 2. The cosmetic sheet product according to item 1, wherein the hydrophobically modified alkyl cellulose is stearoxyhydroxypropylmethyl cellulose.
Item 3. The cosmetic sheet product according to item 1 or 2, wherein the content of the hydrophobically modified alkyl cellulose contained in the solution (I) is 0.01 to 0.9% by weight.
Item 4. The cosmetic sheet product according to any one of items 1 to 3, wherein the cosmetic sheet is used as a face pack sheet.
Item 5. The cosmetic sheet product according to any one of items 1 to 4, wherein in the sheet substrate (II), the sheet substrate carrying a carbonate is a nonwoven fabric.
Item 6. The cosmetic sheet product according to any one of items 1 to 5, wherein in a two-agent type container having two storage sections existing independently of each other with an isolation section capable of being collapsed under an external force, interposed therebetween, the solution (I) is stored in one storage section and the sheet substrate (II) is stored in the other storage section.
Item 7. A cosmetic method including a step of applying a sheet, wherein a sheet substrate (II) carrying a carbonate is impregnated with a solution (I) containing a hydrophobically modified alkyl cellulose, an acid and water, to a body part of a person in need of cosmetic.
Item 8. Use of a solution (I) containing a hydrophobically modified alkyl cellulose, an acid and water and a sheet substrate (I) carrying a carbonate for production of a cosmetic sheet product.

### Advantages of the Invention

According to the present invention, carbon dioxide in the form of minute bubbles can be uniformly and continuously generated when a sheet substrate carrying a carbonate is impregnated with a solution containing an acid. Thus, according to the present invention, carbon dioxide can be efficiently transdermally absorbed, so that various cosmetic effects based on carbon dioxide can be effectively exhibited on the applied skin.

### BRIEF DESCRIPTION OF THE DRAWINGS

FIG. 1 is a view showing a result of observing a state in which a sheet carrying sodium hydrogen carbonate is impregnated with an acid-containing solution (20 seconds after addition of the acid-containing solution) in Example 1.

FIG. 2 is a view showing a result of observing a state in which a sheet carrying sodium hydrogen carbonate is impregnated with an acid-containing solution (20 seconds after addition of the acid-containing solution) in Comparative Example 4.

### EMBODIMENTS OF THE INVENTION

A cosmetic sheet product of the present invention includes: a solution (I) containing a hydrophobically modified alkyl cellulose, an acid and water; and a sheet substrate an carrying a carbonate, and is used with the sheet substrate (II) impregnated with the solution (I). Hereinafter, cosmetic sheet product of the present invention will be described in detail.

### Solution (I)

The solution (I) contains a hydrophobically modified alkyl cellulose. The hydrophobically modified alkyl cellulose is a compound obtained by modifying a water-soluble cellulose ether derivative such as methyl cellulose, ethyl cellulose, propyl cellulose, butyl cellulose, hydroxyethyl cellulose, hydroxypropyl cellulose or hydroxypropylmethyl cellulose with a long-chain alkyl group, and specifically it is represented by the following general formula (1).

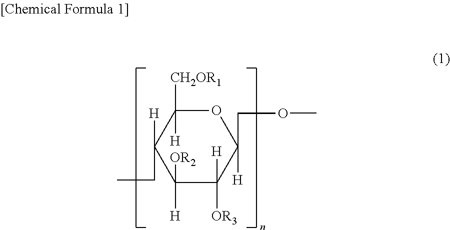

In the general formula (1), R₁, R₂ and R₃ are the same or different, and each represent at least one selected from —[CH₂CH(CH₃)O]ₓ—R₄, —[CH₂CH₂O]_{y}—R₄ and —[CH₂CH(OH)CH₂O]_{z}—R₄. Here, x, y and z are the same or different, and each represent an integer of 0 to 4.

R₄ represents at least one group selected from a hydrogen atom, a linear or branched alkyl group with a carbon number of 1 to 4, and a linear alkyl group with a carbon number of 10 to 28, and R₄ on at least one site in the general formula (1) is a linear alkyl group with a carbon number of 10 to 28. The linear alkyl group with a carbon number of 10 to 28 is preferably a linear alkyl group with a carbon number of 12 to 22,

In the general formula (1), n represent an integer of 200 to 200000.

In the present invention, the hydrophobically modified alkyl celluloses may be used alone, or may be used in combination of two or more thereof.

Among these hydrophobically modified alkyl celluloses, hydroxypropylmethyl cellulose containing a linear alkyl group with a carbon number of 10 to 28 is preferred, with stearoxyhydroxypropylmethyl cellulose being further preferred, from the viewpoint of further effectively achieving uniform and easy mixing of the gel composition (I) and the gel composition (II), uniform generation of carbon dioxide, and prevention of dripping in application to skin,

When stearoxyhydroxypropylmethyl cellulose is used, a preferred example thereof is one in which among hydroxyl groups of a glucose residue as a constituent unit, of the cellulose, 20 to 40%, preferably 27 to 30% of the hydroxyl groups are replaced by methoxyl groups; 5 to 20%, preferably 7 to 11% of the hydroxyl groups are replaced by hydroxypropoxyl groups; and 0.1 to 1.5%, preferably 0.3 to 0% of the hydroxyl groups are replaced by stearyloxyhydroxypropoxyl groups.

As the hydrophobically modified alkyl celluloses, one produced in accordance with a known production method may be used, or a commercial product may be used. For example, stearoxyhydroxypropylmethyl cellulose is commercially available as trade names “SANGELOSE 90L”, “SANGELOSE 90M” and “SANGELOSE 60L” (each manufactured by Daido Chemical Corporation).

While the content of the hydrophobically modified alkyl cellulose in the solution (I) is not particularly limited as long as it is in the form of a liquid, and the object of the present invention can be achieved, the content of the hydrophobically modified alkyl cellulose in the solution (I) is, for example, 0.01 to 0.9% by weight, preferably 0.05 to 0.8% by weight, further preferably 0.05 to 0.7% by weight, especially preferably 0.1 to 0.5% by weight. When the content of the hydrophobically modified alkyl cellulose in the solution (I) falls within the above-mentioned range, the solution (I) can be made to have a suitable liquid form, so that the sheet substrate (II) can be more suitably impregnated with the solution (D. Accordingly, foam in the form of minute bubbles can be uniformly and continuously generated more effectively from the cosmetic sheet product of the present invention. When the content of a hydrophobically modified alkyl cellulose in the solution (I) is excessively high, the solution (I), it may be difficult to impregnate the sheet substrate WO with the solution (I) because the solution (D comes into a gel form.

The solution (I) contains an acid. The acid to be used in the solution (I) is not particularly limited as long as it is a water-soluble and fragrantly acceptable acid, and the acid may be an organic acid or an inorganic acid. Specific examples of the organic acid for use in the present invention include linear fatty acids such as formic acid, acetic acid, propionic acid, butyric acid and valeric acid; dicarboxylic acids such as oxalic acid, malonic acid, succinic acid, glutaric acid, adipic acid, pimelic acid, fumaric acid, maleic acid, phthalic acid, isophthalic acid and terephthalic acid; acidic amino acids such as glutamic acid and aspartic acid; and oxy acids such as glycolic acid, malic acid, tartaric acid, citric acid, lactic acid, hydroxyacrylic acid, α-oxybutyric acid, glyceric acid, tartronic acid, salicylic acid, gallic acid, tropic acid, ascorbic acid and gluconic acid. Specific examples of the inorganic acid for use in the present invention include phosphoric acid potassium dihydrogen phosphate, sodium dihydrogen phosphate, sodium sulfite, potassium sulfite, sodium pyrosulfite, potassium pyrosulfite, acidic sodium hexametaphosphate, acidic potassium hexametaphosphate, acidic sodium pyrophosphate, acidic potassium pyrophosphate and sulfamic acid. These acids may be used alone, or may be used in combination of two or more thereof.

Among these acids, citric acid, malic acid and ascorbic acid are preferred. Above all, acids related to in vivo TCA cycles (citric acid, malic acid and the like) acts to further enhance the cosmetic effect, and is suitably used in the present invention.

While the content of the acid in the solution (I) is appropriately set according to a type of the acid, an amount of the carbonate contained in the sheet substrate (II) described later, and so on, the content of the acid in the solution (I) is, for example, 0.1 to 10% by weight, preferably 0.5 to 5% by weight, further preferably 1 to 2% by weight.

The solution (I) contains water. Water to be used in the solution (I) may be, for example, purified water, distilled water, ion-exchanged water, tap water, ultrapure water, membrane-filtered water or the like.

The content, of water in the solution (I) is not particularly limited as long as the solution (I) can form a liquid, the content of the water is, for example, 70 to 99.9% by weight, preferably 80 to 99.5% by weight, further preferably 90 to 99% by weight.

The solution (I) may contain, in addition to the above-described components, other components capable of being blended in a cosmetic and an external pharmaceutical product as necessary. Examples of other components capable of being blended as described above include skin whitening agents, astringents, moisturizing agents, anti-inflammatory agents, ultraviolet absorbers, antiperspirants, plant extracts, amino acids, vitamins, alcohols, fat-and-oil surfactants, thickeners other than hydrophobically modified alkyl celluloses, antioxidants, chelating agents, antihistamines, sebum inhibitors, antiseborrheic agents, antipruritic agents, alcohols, fatty-and-oil components, surfactants, antioxidants, preservatives, germicides, chelating agents, flavors and pigments.

While the pH of the solution (I) is determined according to a type and an amount of the contained acid, the pH of the gel composition (I) is normally 1 to 6.5, preferably 1 to 5.5, further preferably 1.5 to 4.

### Sheet Substrate (II)

The sheet substrate (II) carries a carbonate. While the carbonate to be used in the sheet substrate (II) is not particularly limited as long as it is water-soluble, and can produce carbon dioxide by reacting with an acid in the presence of water, examples thereof include ammonium carbonate, potassium carbonate, sodium carbonate, sodium hydrogen carbonate, potassium sesquicarbonate, calcium sesquicarbonate, sodium sesquicarbonate and potassium hydrogen carbonate.

These carbonates may be used alone, or may be used in combination of two or more thereof.

Among these carbonates, sodium hydrogen carbonate is preferred.

While the carried amount of the carbonate in the sheet substrate (II) is appropriately set according to a type of the carbonate, an amount and type of the acid contained in the solution (I) described above, and so on, the carried amount of the carbonate in the sheet substrate (II) is, for example, 0.1 to 100 g, preferably 1 to 50 g, further preferably 5 to 30 g per 1 m² of the sheet substrate.

The raw material of the sheet substrate carrying a carbonate is not particularly limited, and may be natural fiber, chemical fiber or a combination thereof. Specific examples of the natural fiber include cotton, pulp, kapok, hemp, jute, ramie, coconut, Areca catechu, wool and silk. Examples of the chemical fiber include rayon, nylon, vinylon, acetate, acryl, polyester, polyethylene, polypropylene, polyurethane and polyamide. These raw materials may be used alone, or may be used in combination of two or more thereof. Among them, from the viewpoint of sense of use, etc., cotton, pulp, rayon, wool and silk are preferred, with cotton, pulp and rayon being further preferred.

The sheet substrate carrying a carbonate may be any of a woven fabric, a nonwoven fabric, a nonwoven fabric laminate, a cotton-like sheet, a porous sheet and the like, but a nonwoven fabric is preferred from the sense of use, etc.

While the basis weight of the sheet substrate carrying a carbonate is not particularly limited, the basis weight of the sheet substrate carrying a carbonate is, for example, 0.1 to 300 g/m², preferably 10 to 150 g/m².

The shape of the sheet substrate carrying a carbonate is not particularly limited as long as it is a shape conforming to a skin part to which the cosmetic sheet product is applied. For example, in the case of use as a face pack sheet, it is only necessary that the sheet substrate be formed into a shape conforming to a facial shape,

The method for causing the sheet substrate to carry a carbonate is not particularly limited, and for example, a known method such as an immersion method, a spraying method or a coating method can be employed to cause the sheet substrate to carry a carbonate. For example, the sheet substrate is caused to carry a carbonate by an immersion method, the sheet substrate is immersed in water with a carbonate dissolved therein (preferably water with a carbonate dissolved therein at a saturated concentration), and then dried.

### Use Mode

The cosmetic sheet product of the present invention is applied to skin as a cosmetic sheet product containing carbon dioxide with carbon dioxide generated on the sheet substrate by impregnating the sheet substrate (II) with the solution (I) at the time of use,

The amount of the solution (I) caused to impregnate the sheet substrate (II) is appropriately set according to a composition of the solution (I), an amount of the carbonate carried on the sheet substrate (II), and so on, and may be set so that the amount of the solution (I) is normally 0.5 to 50 ml, preferably 1 to 20 ml, further preferably 2 to 10 ml per 1 g of the sheet substrate (II).

For the cosmetic sheet product of the present invention, the container for storing the solution (I) and the sheet substrate (II) is not particularly limited as long as it is designed so that the solution (I) and the sheet substrate (II) are separately stored, and the sheet substrate (II) can be impregnated with the solution (I) at the time of use, but a two-agent type container having two storage sections existing independently of each other with an isolation section, which can be collapsed under an external force such as an external pressure, interposed therebetween is preferred from the viewpoint of use convenience. When such a two-agent type container is used, the solution (I) is stored in one storage section while the sheet substrate (II) is stored in the other storage section, and the isolation section isolating the solution (I) and the sheet substrate (II) from each other at the time of use, so that the sheet substrate (II) can be impregnated with the solution (I).

The cosmetic sheet product of the present invention is used as a sheet pack cosmetic. The cosmetic sheet product of the present invention is capable of uniformly and continuously carbon dioxide and causing carbon dioxide to be efficiently transdermally absorbed, and therefore various cosmetic effects based on carbon dioxide can be effectively exhibited.

The body part to which the cosmetic sheet product of the present invention is not particularly limited, and examples thereof include facial areas such as a forehead, an area around the eye, a corner of the eye, a cheek, a jaw and a lip as well as an elbow, an arm, a fingertip, a leg, a knee, an abdomen, a flank, a neck, a back and the like. Above all, the cosmetic sheet product of the present invention is suitable as a face pack sheet that is applied to the face.

The cosmetic sheet product of the present invention can be used for the purpose of, for example, improvement of skin quality such as amelioration of rough skin; slimming of a body part such as a face, a leg, an arm, an abdomen, a flank, a back, a neck or a jaw (including amelioration of flabbiness, tightening of skin, etc.); or whitening, humidification, softening of skin, amelioration of darkening, amelioration of stains, amelioration of freckles, or the like.

While the use frequency/time of the cosmetic sheet product of the present invention may be appropriately set according to a use purpose, the cosmetic sheet product is applied, for example, for about 5 to 30 minutes per one time with a frequency of one time per 1 to 14 days.

### EXAMPLES

The present invention will be described in detail below by showing examples, but the present invention is not intended to be limited to these examples.

### Test Example

A nonwoven fabric formed into a a facial shape (“KP9560” (manufactured by Sanshoshigyo Co., Ltd.); a mixture of 35% of rayon, 45% of pulp and 10% of PET; basis amount 60 g/m²; circular shape with a diameter of 20 cm) was immersed in an aqueous solution containing sodium hydrogen carbonate at a concentration of 8.3% by weight, then taken out, and dried at about 100° C. to prepare a sheet carrying sodium hydrogen carbonate. On the other hand, acid-containing solutions of compositions shown in Tables 1 and 2 were prepared.

A two-agent type container having two storage sections existing independently of each other with an isolation section, which could be collapsed by pressing with a finger, interposed therebetween, was provided, the sheet carrying sodium hydrogen carbonate was folded to a size equivalent to about 1/12 of its original size and stored in one of the storage sections, and 20 ml of the acid-containing solution was stored in the other storage section. Then, the storage section storing the acid-containing solution was pressed with a finger to collapse the isolation section, so that the sheet carrying sodium hydrogen carbonate was impregnated with the acid-containing solution, and the sheet was taken out from the container 20 seconds after the isolation section was collapsed. For the state when the sheet, carrying sodium hydrogen carbonate was impregnated with the acid-containing solution (compatibility state, foaming state, and foaming state in the sheet taken out from the container), evaluations were performed in accordance with the following assessment criteria.

<Evaluation of State when Sheet Carrying Sodium Hydrogen Carbonate is Impregnated with Acid-Containing Solution>
0: It is difficult for the acid-containing solution to move to the storage section storing the sheet carrying sodium hydrogen carbonate.
1: The acid-containing solution can move to the storage section storing the sheet carrying sodium hydrogen carbonate. However, the sheet carrying sodium hydrogen carbonate is not uniformly impregnated with the acid-containing solution.
2: The acid-containing solution can easily move to the storage section storing the sheet carrying sodium hydrogen carbonate. Carbon dioxide in the form of large bubbles is generated, and bubbles of carbon dioxide do not remain when the sheet is taken out from the container.
3: The acid-containing solution can easily move to the storage section storing the sheet carrying sodium hydrogen carbonate. Carbon dioxide in the form of large bubbles is generated, and existence of bubbles of carbon dioxide on the sheet can be confirmed immediately after the sheet is taken out from the container, but bubbles of carbon dioxide on the sheet disappear after 5 minutes.
4: The acid-containing solution can easily move to the storage section storing the sheet carrying sodium hydrogen carbonate. Carbon dioxide in the form of minute bubbles is uniformly generated, and existence of bubbles of carbon dioxide on the sheet can be confirmed immediately after the sheet is taken out from the container, but bubbles of carbon dioxide on the sheet disappear after 5 minutes.
5: The acid-containing solution can easily move to the storage section storing the sheet carrying sodium hydrogen carbonate. Carbon dioxide in the form of minute bubbles is uniformly generated, and existence of bubbles of carbon dioxide on the sheet can be confirmed both immediately after and 5 minutes after the sheet is taken out from the container.

Further, the isolation section of the two-agent type container was collapsed, so that the sheet carrying sodium hydrogen carbonate was impregnated with the acid-containing solution. Twenty (20) seconds after the isolation section was collapsed, the sheet was taken out from the container, and brought into close contact with the inner side of the front arm with the sheet folded to a size equivalent to ½ of its original size. One (1) minute, 5 minutes and 10 minutes thereafter, a level of reddening of skin at the inner side of the front arm, which the sheet was brought into close contact with, was evaluated in accordance with the following assessment criteria. Reddening of skin at a sheet contact part indicates that carbon dioxide was absorbed to promote blood circulation,

### <Level of Reddening of Skin at Sheet Contact Part>

1: Skin is not reddened,
2: Skin is partially reddened.
3: Skin is reddened as a whole, but the shape of the reddened region is not clear.
4: Reddening is observed, and it is somewhat recognized that the shape of the reddened region is identical to the shape of the sheet.
5: Reddening is observed, and it is clearly recognized that the shape of the reddened region is identical to the shape of the sheet.

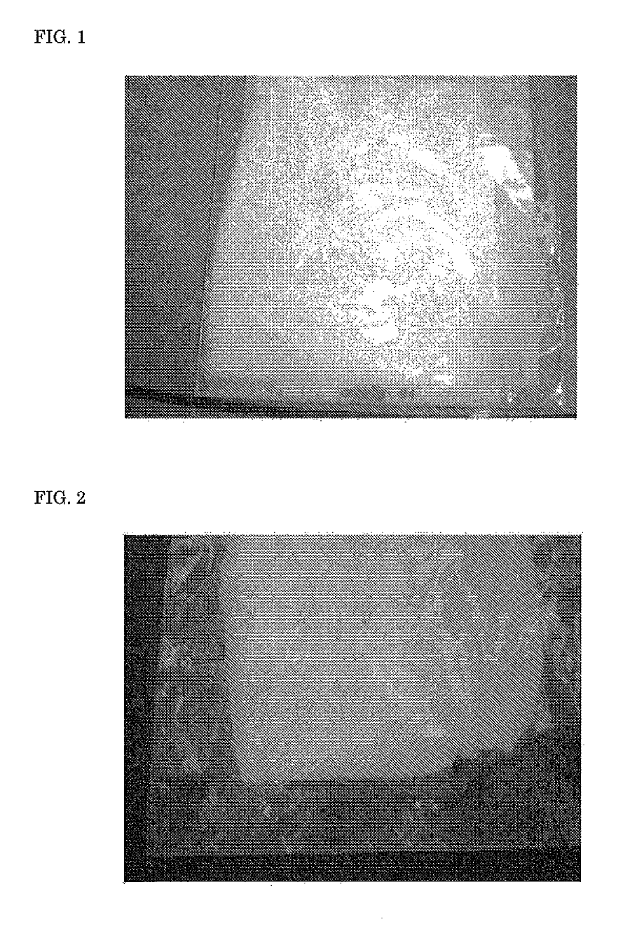

The obtained results are collectively shown in Tables 1 and 2. From these results, it has been confirmed that in the case where stearoxyhydroxypropylmethyl cellulose was blended in the acid-containing solution, carbon dioxide in the form of minute bubbles could be uniformly and continuously generated. It has become evident that in the case where stearoxyhydroxypropylmethyl cellulose was blended in the acid-containing solution, carbon dioxide was efficiently transdermally absorbed because sufficient reddening was observed when the sheet was applied to skin. On the other hand, in the case where a thickener other than stearoxyhydroxypropylmethyl cellulose was used in the acid-containing solution, only large bubbles of carbon dioxide could be generated, and carbon dioxide could not be continuously generated. In the case where a thickener other than stearoxyhydroxypropylmethyl cellulose was used in the acid-containing solution, sufficient reddening was not observed when the sheet was applied to skin. For reference, the result of observing a state in which the sheet carrying sodium hydrogen carbonate is impregnated with the acid-containing solution (20 seconds after addition of the acid-containing solution) in Example 1 is shown in FIG. 1, and the result of observing a state in which the sheet carrying sodium hydrogen carbonate is impregnated with the acid-containing solution (20 seconds after addition of the acid-containing solution) in Comparative Example 4 is shown in FIG. 2.

TABLE 1

| | | Examples | | | | Comparative Examples | | | | | |
|---|---|---|---|---|---|---|---|---|---|---|---|
| | | 1 | 2 | 3 | 4 | 1 | 2 | 3 | 4 | 5 | 6 |
| Citric acid | | 1.5 | 1.5 | 1.5 | 1.5 | 1.5 | 1.5 | 1.5 | 1.5 | 1.5 | 1.5 |
| Stearoxyhydroxypropylmethyl | | 0.4 | 0.5 | 0.1 | 0.1 | — | — | — | — | — | — |
| cellulose^{#1} | | | | | | | | | | | |
| Sodium alginate | | — | — | — | — | 0.5 | 1.0 | — | 0.2 | — | — |
| Cellulose gum | | — | — | — | 0.2 | — | — | 0.5 | 0.1 | — | — |
| Carrageenan | | — | — | — | 0.1 | — | — | — | 0.2 | — | — |
| Xanthan gum | | — | — | — | — | — | — | — | — | 0.5 | 0.25 |
| Locust bean gum | | — | — | — | — | — | — | — | — | — | 0.25 |
| Water | | Balance | Balance | Balance | Balance | Balance | Balance | Balance | Balance | Balance | Balance |
| Evaluation of state when | | 5 | 5 | 5 | 5 | 2 | 3 | 2 | 2 | 2 | 3 |
| sheet carrying sodium | | | | | | | | | | | |
| hydrogen carbonate is | | | | | | | | | | | |
| impregnated with | | | | | | | | | | | |
| acid-containing solution | | | | | | | | | | | |
| Level of reddening | after 1 | 5 | 5 | 5 | 5 | 4 | 4 | 4 | 4 | 2 | 4 |
| of skin at sheet | minute | | | | | | | | | | |
| contact part | after 5 | 5 | 5 | 4 | 4 | 2 | 3 | 2 | 2 | 2 | 2 |
| | minutes | | | | | | | | | | |
| | after 10 | 3 | 4 | 2 | 3 | 1 | 2 | 1 | 1 | 2 | 1 |
| | minutes | | | | | | | | | | |

In the table, the unit of the content of each blended component is “% by weight”,
^{#1}As stearoxyhydroxypropylmethyl cellulose, “SANGELOSE 60L” (trade name) (manufactured by Daido Chemical Corporation) was used.

TABLE 2

| | | Comparative Examples | | | |
|---|---|---|---|---|---|
| | | 7 | 8 | 9 | 10 |
| Citric acid | | 1.5 | 1.5 | 1.5 | 1.5 |
| Carrageenan | | — | 0.1 | — | — |
| Xanthan gum | | — | — | 1.0 | — |
| (Acryloyl dimethyl taurine | | — | — | — | 1.0 |
| ammonium/VP) copolymer | | | | | |
| (Carboxyethyl acrylate/acryloyl | | 3.0 | — | — | — |
| dimethyl taurine Na) copolymer | | | | | |
| Water | | Balance | Balance | Balance | Balance |
| Evaluation of state when sheet | | 2 | 2 | 2 | 2 |
| carrying sodium hydrogen | | | | | |
| carbonate is impregnated with | | | | | |
| acid-containing solution | | | | | |
| Level of reddening | after | 3 | 3 | 3 | 3 |
| of skin at sheet | 1 minute | | | | |
| contact part | after | 3 | 2 | 2 | 3 |
| | 5 minutes | | | | |
| | after | 2 | 1 | 1 | 2 |
| | 10 minutes | | | | |

In the table, the unit of the content of each blended component is “% by weight”.

1. A cosmetic sheet product, comprising a solution (I) containing a hydrophobically modified alkyl cellulose, an acid and water; and a sheet substrate (II) carrying a carbonate and being used with the sheet substrate (II) impregnated with the solution (I).

2. The cosmetic sheet product according to claim 1, wherein the hydrophobically modified alkyl cellulose is stearoxyhydroxypropylmethyl cellulose.

3. The cosmetic sheet product according to claim 1, wherein the content of the hydrophobically modified alkyl cellulose contained in the solution (I) is 0.01 to 0.9% by weight.

4. The cosmetic sheet product according to claim 1 to 3, wherein the cosmetic sheet is used as a face pack sheet.

5. The cosmetic sheet product according to claim 1, wherein in the sheet substrate (II), the sheet substrate carrying a carbonate is a nonwoven fabric.

6. The cosmetic sheet product according to claim 1, wherein in a two-agent type container having two storage sections existing independently of each other with an isolation section capable of being collapsed under an external force, interposed therebetween, the solution (I) is stored in one storage section and the sheet substrate (II) is stored in the other storage section.

7. A cosmetic method comprising a step of applying a sheet, wherein a sheet substrate (II) carrying a carbonate is impregnated with a solution (I) containing a hydrophobically modified alkyl cellulose, an acid and water, to a body part of a person in need of cosmetic.

8. Use of a solution (I) containing a hydrophobically modified alkyl cellulose, an acid and water and a sheet substrate (II) carrying a carbonate for production of a cosmetic sheet product.
